(19) United States
(12) Patent Application Publication
(10) Pub. No.: US 2011/0132379 A1
(43) Pub. Date: Jun. 9, 2011

(54) Head-Shape Remodelling Instrument

(76) Inventor: Young Joon Lee, Seoul (KR)

(21) Appl. No.: 12/863,552

(22) PCT Filed: Jan. 16, 2009
(86) PCT No.: PCT/KR09/00239

§ 371 Date: Aug. 30, 2010

(30) Foreign Application Priority Data
Jan. 18, 2008 (KR) 10-2008-0005556

(51) Int. Cl. A61F 5/00

(52) U.S. Cl. 128/857

(57) ABSTRACT

A cranial orthosis for infants, which can not only prevent the brachycephaly and plagiocephaly but also correct a brachycephalic head into a scaphocephalic head, is provided. The cranial orthosis includes a protective outer cover that is formed of a hard material and having an inner surface formed in a scaphocephalic shape to cover at least an occipital region and sides of a head, and a soft inner cover that is formed of a soft material, shaped to correspond to the protective cover, and mounted on an inner surface of the protective outer cover. The soft inner cover is formed to be spaced apart from a peak portion of the occipital region.

### CROSS-REFERENCE TO RELATED APPLICATIONS

The present application claims priority under 35 U.S.C. 119 and 35 U.S.C. 365 to Korean Patent Application No. 10-2008-0005556 (filed on Jan. 18, 2008), which is hereby incorporated by reference in its entirety.

### BACKGROUND

The present disclosure relates to a cranial orthosis for correcting cranium and facial bone shapes in infants.

In more detail, the present disclosure relates to a cranial orthosis for infants, which can not only prevent the brachycephaly and plagiocephaly but also correct a brachycephalic head into a scaphocephalic head.

Human cranium shapes can be classified into a scaphocephalic shape that is long in a frontward-backward direction and short in a leftward-rightward direction and a brachycephalic shape that is long in the leftward-rightward direction and short in the frontward-backward direction. Generally, the westerner people have the scaphocephalic head and the oriental people have the brachycephalic head.

With the recent preference of the oriental people for the scaphocephalic head, the parents try to put their baby to sleep in a state where the baby lies on his/her stomach with his/her head turning to a side so that his/her head can be naturally formed in the scaphocephalic shape through pressure applied to the side of his/her head by the weight of the head. However, the recent research shows that the sleeping of the baby lying on his/her stomach may be the trigger for baby's sudden death. Therefore, medical professionals alert the parents about the dangers of correction of the cranium by the position of the body. That is, the medical professionals encourage the parents to put their baby (infant) to sleep in a state where the baby lies face up.

Meanwhile, for infants who are born with the plagiocephaly or suffer from the plagiocephaly by an accident, some medical centers have used a cranial orthosis helmet that is specifically manufactured as a medical device.

Referring to FIG. 17 showing photographs of a cranium, it can be noted that the cranium of an infant is skew before he/she wears the helmet but, after the infant wears the helmets for several months or more, the cranium of the infant has bilateral symmetry.

The cranial orthosis helmet is made of a hard material to correspond to a desired head shape and worn on the head of the infant to actively press the head until the head of the infant is corrected to the desired head shape. The cranial orthosis helmet is generally designed to actively press an abnormal portion of the cranium and used as a medical supportive device after the treatment of the plagiocephaly or the correcting operation of the cranium.

Meanwhile, most of the cranial orthosis helmets are designed to prevent a rear head of the infant from being flat by actively pressing the head of the infant and are formed not to make the infant sleep obliquely and thus to prevent a rear head shape from being asymmetrical.

However, according to the above-described cranial orthosis methods, the hard cranial orthosis helmet that is designed to actively press the head provokes controversy that organs such as a brain of the head may not be normally grown. In addition, the hard helmet pressing the cranium may cause the secondary plagiocephaly. Furthermore, since the helmet is worn on the head to cover an entire portion of the head, the user may have a heavy feeling and the ventilation is deteriorated. When children after the infantile wear the helmet, the correction must be performed over the long term and the correction effect cannot be satisfactorily attained. Further, the helmet is very expensive ($2,000-$4,000). This would make the cost burden too heavy for the user.

Meanwhile, U.S. Pat. Nos. 5,951,503 A and 6,939,316 B2 disclose a cranial orthosis band that is developed for western infants having a scaphocephalic head which is long in a frontward-backward direction and short in a leftward-rightward direction. That is, as shown in FIGS. 18 to 20 (for descriptive convenience, the drawings and the numbers in the original patent documents are used), the cranial orthosis band of the patents includes an exterior covering 12 and a plurality of fabric layers 48, 50, 52, and 54 covering an interior covering, and a wing portion 14 that can be supportably fixed on both sides of the cranium.

However, as shown in FIGS. 19 and 20, when the infant lies on his/her back on a mattress in a state where he/she wears the cranial orthosis, a peak portion of an occipital region of the infant directly contacts the cranial orthosis. In addition, when the infant lies obliquely on the mattress, the peak portion of the occipital region of the infant still contacts the cranial orthosis. Further, both sides of the occipital region between the both sides of the head which contact the wing portion 14 and the peak portion of the occipital region do not directly contact the cranial orthosis. In addition, predetermined gaps 44 remain between the both sides of the occipital region and the cranial orthosis.

That is, the cranial orthosis of the U.S. patents is designed for the western people mostly having the scaphocephalic head, and the cranial orthosis of the U.S. patents are designed for the purpose of prevention and correction of the plagiocephaly. Therefore, the cranial orthosis of the U.S. patents is not proper for prevention and correction of the brachycephaly.

### SUMMARY

Embodiments provide a cranial orthosis that can prevent the brachycephaly where an occipital region is flat and the plagiocephaly where the occipital region is deformed by preventing a peak portion of the occipital region from being pressed by contacting regardless of a lying posture of an infant (for example, the infant lies on his/her back or obliquely).

Embodiments also provide a cranial orthosis that is designed such that both sides of an occipital region and sides of the head except for a peak portion of the occipital region can pressed by a weight of a wearer's head, thereby naturally correcting a brachycephalic head into a scaphocephalic head.

Embodiments also provide a cranial orthosis that can maintain a stable wearing state while reducing a weight thereof by being designed in a semi-helmet shape and can improve a wearing feeling by reducing a heavy feeling of the wearer and improving ventilation.

Embodiments also provide a cranial orthosis that is designed to prevent a sudden death of an infant by eliminating necessary for putting the infant sleep on his/her stomach.

In one embodiment, a cranial orthosis includes: a protective outer cover that is formed of a hard material and having an inner surface formed in a scaphocephalic shape to cover at least an occipital region and sides of a head; and a soft inner cover that is formed of a soft material, shaped to correspond to the protective outer cover, and mounted on an inner surface of the protective outer cover, wherein the soft inner cover is formed to be spaced apart from at least a peak portion of the occipital region.

The soft inner cover may be formed to contact the sides of the head and side of the occipital region except for the peak portion. At this point, the head contacting portion may be thicker than other portions.

The soft inner cover may include a middle part that does not contact the head and side parts provided at both sides of the middle part and contacting the head.

A ventilation hole may be formed through the protective outer cover and the soft inner cover. At this point, the ventilation hole may be formed at a location spaced apart from the occipital region to maximize the ventilation effect.

The cranial orthosis may further include arm portions extending frontward from both ends of the protective outer cover; a head fixing member formed above the protective outer cover and closely contacting a top of the head to prevent the cranial orthosis from moving downward; and a jaw hooking member formed under the protective outer cover and hooked around a jaw to prevent the cranial orthosis from moving upward.

At this point, the head fixing member and the jaw hooking member may be provided in the form of an elastic band so that they can be more securely fixed.

A rear end portion of the protective outer cover may be flat to prevent the head of the infant lying on the floor from moving.

The protective outer cover may be is divided into a plurality of sections considering a formability and design of a product.

The details of one or more embodiments are set forth in the accompanying drawings and the description below. Other features will be apparent from the description and drawings, and from the claims.

### BRIEF DESCRIPTION OF THE DRAWINGS

FIG. 1 is a perspective view of a cranial orthosis according to a first embodiment.

FIG. 2 is a view illustrating a state where an infant wearing the cranial orthosis of FIG. 1 lies on his/her back on a floor (mattress).

FIG. 3 is a view illustrating a state where the infant wearing the cranial orthosis of FIG. 1 lies obliquely.

FIG. 4 is a view illustrating an infant wearing the cranial orthosis of FIG. 1.

FIG. 5 is a view illustrating a state where an infant wearing a cranial orthosis according to a second embodiment lies on his/her back on a floor (mattress).

FIG. 6 is a view illustrating a state where the infant wearing the cranial orthosis of FIG. 5 lies obliquely.

FIG. 7 is a perspective view of a cranial orthosis according to a third embodiment.

FIG. 8 is a view illustrating a state where an infant wearing the cranial orthosis of FIG. 7 lies on his/her back on a floor (mattress).

FIG. 9 is a view illustrating a state where the infant wearing the cranial orthosis of FIG. 8 lies obliquely.

FIG. 10 is a perspective view of a cranial orthosis according to a fourth embodiment.

FIG. 11 is a front view of the cranial orthosis of FIG. 10.

FIG. 12 is a side view of the cranial orthosis of FIG. 10.

FIG. 13 is a view illustrating a separating state of the cranial orthosis of FIG. 10.

FIG. 14 is a view illustrating a state where an infant wearing the cranial orthosis of FIG. 10 lies on his/her back on a floor (mattress).

FIG. 15 is a view illustrating a state where the infant wearing the cranial orthosis of FIG. 14 lies obliquely.

FIG. 16 is a perspective view of a cranial orthosis according to a fifth embodiment.

FIG. 17 illustrates photographs showing a cranium before and after wearing a cranial orthosis.

FIG. 18 is a perspective view of a cranial orthosis for an infant according to a related art.

FIG. 19 is a view illustrating a state where an infant wearing the cranial orthosis of FIG. 18 lies on his/her back on a floor (mattress).

FIG. 20 is a view illustrating a state where the infant wearing the cranial orthosis of FIG. 18 lies obliquely.

### DETAILED DESCRIPTION OF THE EMBODIMENTS

Reference will now be made in detail to the embodiments of the present disclosure, examples of which are illustrated in the accompanying drawings.

For the understanding of the embodiments, separate reference numbers will refer to a protective outer cover 10, an arm portion 12, and a soft inner cover 30 in the respective embodiments.

A cranial orthosis according to the embodiments is an inventive semi-helmet type cranial orthosis that can effectively protect a cranium deformation in infants, such as deformation into a brachycephalic head shape that is wide in a leftward-rightward direction and short in a frontward-backward direction. Particularly, the embodiments relate to a semi-helmet type cranial orthosis for infants that can passively safely form a scaphocephalic head shape that is long in the frontward-backward direction and narrow in the leftward-rightward direction.

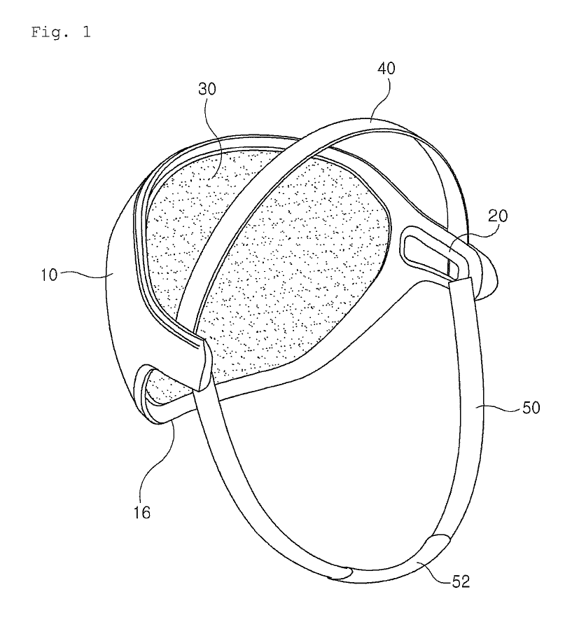

That is, the cranial orthosis is designed in a semi-helmet type that structurally encloses an occipital region or sides of the head so as to prevent not only the plagiocephaly where the cranium is skew in a side but also the brachycephaly where the occipital region is flat and to correct the brachycephalic head shape into the scaphocephalic head shape. In more detail, the cranial orthosis is designed in the semi-helmet type formed in the scaphocephalic shape such that a central portion of the cranial orthosis does not directly contact a peak portion of the occipital region but only peripheral portion of the central portion presses the sides of the occipital region. That is, the cranial orthosis of the present invention can be formed in a variety of shapes shown in FIGS. 1, 5, 7, 10 and 16.

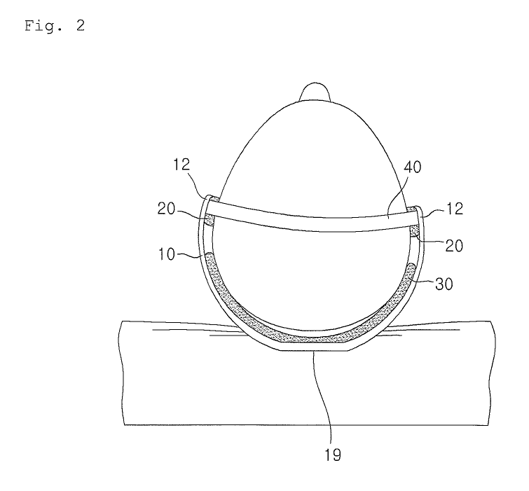

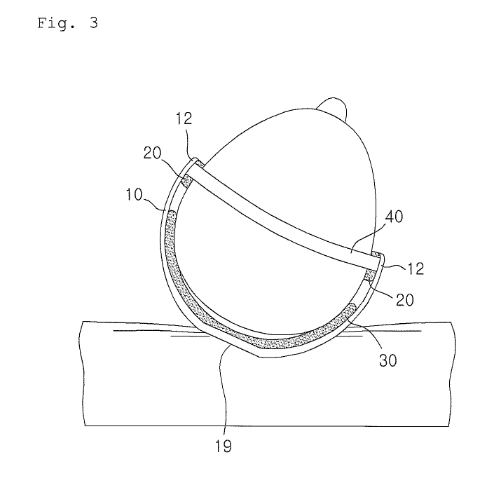

FIG. 1 is a perspective view of a cranial orthosis according to a first embodiment, FIG. 2 is a view illustrating a state where an infant wearing the cranial orthosis of FIG. 1 lies on his/her back on a floor (mattress), FIG. 3 is a view illustrating a state where the infant wearing the cranial orthosis of FIG. 1 lies obliquely, and FIG. 4 is a view illustrating an infant wearing the cranial orthosis of FIG. 1.

Referring to FIGS. 1 to 4, a cranial orthosis according to an embodiment includes a protective outer cover 10 that is formed of a hard material so that a shape of a cranium is changed, a soft inner cover 30 (30a, 30b, 30c) that functions as an absorber between the protective outer cover 10 and a head of the infant wearing the cranial orthosis, and a head fixing member 40 closely contacting an upper portion of the head to prevent the cranial orthosis from being separated from the head. If necessary, the cranial orthosis may further include a jaw hooking member 50 that is hooked on a jaw of the infant so that the cranial orthosis can be more stably worn.

The cranial orthosis may further include arm portions 12 that are formed on both side ends of the protective outer cover 10 so that the cranial orthosis can be stably supported on the head of the infant. The head fixing member 40 is coupled to outer sides of the arm portions 12. Further, a gap maintaining member 20 is attached to inner sides of the arm portions 12 so that the cranial orthosis can stably contact the both sides of the head of the infant.

In more detail, the protective outer cover 10 is formed of the hard material and shaped to correspond to a normal head of the infant. The protective outer cover 10 is formed in the semi-helmet shape that does not entirely press the head of the infant but press only the sides of the head and the occipital region.

In more detail, the protective outer cover 10 is a portion that directly contacts a floor (mattress) when the infant sleeps on the floor. The protective outer cover 10 is formed of the hard material so that it can press the head using a weight of the head of the infant regardless of the lying posture of the infant.

Further, an inner surface of the protective outer cover 10 is shaped to correspond to a scaphocephalic head shape that is symmetrical vertically and gradually narrowed rearward so as to prevent the head of the infant from being flat and deformed and to form the head of the infant in the scaphocephalic head shape. The inner surface of the protective outer cover 10 is designed to contact only the sides of the head and the occipital region. That is, at least the inner surface of the protective outer cover 10 is designed to correspond to a head shape of the scaphocephalic infant.

In addition, the arm portions 12 on which ears of the infant are supported are formed on the both front-side ends of the protective outer cover 10.

Meanwhile, the soft inner cover 30 is formed of an elastic, soft material and shaped to correspond to the protective outer cover 10. The soft inner cover 30 is closely coupled to the inner surface of the protective outer cover 10.

In more detail, the soft inner cover 30 is formed to contact the sides of the head and the occipital region except for a peak portion of the occipital region. The soft inner cover 30 is formed in an oval scaphocephalic head shape. That is, an empty space is formed between the peak portion of the occipital region of the infant and the soft inner cover 30.

At this point, the empty portion between the peak portion of the occipital region of the infant and the soft inner cover 30 may be formed by the shape of the protective outer cover 10 or the shape of the soft inner cover 30.

In this embodiment, the soft inner cover 30 is monolithic and coupled to the interior side of the protective outer cover 10. That is, the soft inner cover 30 corresponds to the shape of the protective outer cover 10 and is monolithic and coupled to the interior surface of the protective outer cover 10.

Meanwhile, since the infant sleeps most of his/her day, almost all day, his/her head sweats a lot. Therefore, the soft inner cover 30 is wetted by the sweat and dried. This is repeated. By this, the soft inner cover 30 gives off a bad smell or grows bacteria. Therefore, the soft inner cover 30 is detachably attached to the protective outer cover 10 so that it is separated from the protective outer cover 10 and washed. For example, the soft inner cover 30 is attached to the inner surface of the protective outer cover 10 by a Velcro fastener so that it can be easily detached from the protective outer cover 10.

Meanwhile, the protective outer cover 10 may be provided with a position maintaining portion 19 that is flat so that the cranial orthosis does not shake on the floor. The position maintaining portion 19 is formed on a rear end portion of the protective outer cover 10. When the infant wears the cranial orthosis and lies on his/her back, the position maintaining portion 19 prevents the cranial orthosis from shaking in the leftward-rightward direction and allows the infant to sleep soundly in a stable state.

Further, the arm portions 12 extend frontward from the both side ends of the protective outer cover 30 and are hooked on upper sides of the ears of the infant. As the arm portions 12 are hooked on the ears at the sides of the head of the infant, the cranial orthosis can be worn at an accurate location and supported stably on the head of the infant.

Further, the head fixing member 40 is coupled to the outer sides of the arm portions 12. The head fixing member 40 is formed in a strip shape and contacts a top of the head. That is, opposite end portions of the head fixing member 40 are respectively fixed on the arm portions 12 and a middle portion of the head fixing member 40 closely contacts the top of the head to prevent the cranial orthosis from moving downward. Therefore, the wearing state of the cranial orthosis can be securely maintained.

At this point, the head fixing member 40 may be formed of an elastic band that can be elastically deformed in accordance with a head size of the infant to securely maintain the wearing state.

Further, the gap maintaining members 20 are separately provided on the inner sides of the arm portions 12 so that the cranial orthosis can further closely contact the head of the infant and be comfortably stably supported on the head. The gap maintaining members 20 have a similar thickness to the soft inner cover 30. At this point, instead of separately providing the gap maintaining members 20, the soft inner cover 30 extends to the arm portions 12 to function as the gap maintaining members 20.

The cranial orthosis of this embodiment can be used to prevent the deformation of the head shape or correct the head shape by preventing the head of the infant from being pressed. However, it is preferable that the jaw hooking member 50 formed in a strip shape is additionally provided so as to securely maintain the wearing state by being hooked on a lower portion of the jaw of the infant.

The jaw hooking member 50 is coupled to sides of the arm portions 12. Like the head fixing member 40, the jaw hooking member 50 may be formed of an elastic band that can be elastically deformed in accordance with the head size of the infant to securely maintain the wearing state.

At this point, a coupling maintaining band 52 may be provided on a middle portion of the jaw hooking member 50 so that the jaw hooking member 50 can be smoothly supported on the jaw by the frictional force.

That is, according to the cranial orthosis of this embodiment, the protective outer cover 10 and the soft inner cover 30 closely contact the occipital region of the infant and the gap maintaining members 20 are provided on the inner sides of the arm portions 12 to fix the cranial orthosis on the left and right sides of the head of the infant. Therefore, even when the infant is in activity or lies and moves, the wearing state of the cranial orthosis can be securely maintained.

Further, in order to stably keep the cranial orthosis when it is not used, a flat bottom portion 16 may be provided on a lower end portion of the protective outer cover 10.

The following will describe the operation and effects of the above-described cranial orthosis of the first embodiment.

The protective outer cover 10 is formed of the hard material in the scaphocephalic head shape to function to apply pressure to correct the head shape. The elastic, soft inner cover 30 functions to absorb impact between the protective outer cover 10 and the head of the infant, fix the head of the infant, sock the sweat, and ventilate.

In more detail, referring to FIG. 2, when the infant wearing the cranial orthosis of the first embodiment lies on his/her back, the head is supported on the protective outer cover 10 and the soft inner cover 30. At this point, since the soft inner cover 30 is formed of the elastic, soft material, the pressure is not forcedly directly applied to the head of the infant but natural pressure generated by the weight of the head of the infant is applied to the head.

At this point, the pressure is applied only to the sides of head and the occipital region except for the peak portion. In addition, a contact area of the head with the soft inner cover 30 varies in accordance with the head shape of the infant and thus the protruding portion or skew portion of the head that have a relatively wider contact area with the soft inner cover 30 is applied with relatively higher pressure. Therefore, the asymmetrically deformed head or the brachycephalic head is gradually corrected into the scaphocephalic head that is symmetrical and gentle oval shape in response to the scaphocephalic curve of the protective outer cover 10.

Particularly, the peak portion of the occipital region of the infant remains without contacting any one of the protective outer cover 10 and the soft inner cover 30. That is, the empty space is formed between the peak portion of the occipital region of the infant and the soft inner cover 30.

Accordingly, only the sides of the head and the occipital region except for the peak portion are applied with the pressure formed by the weight of the head of the infant. That is, since there is no pressure applied to the peak portion of the occipital region of the infant, the brachycephaly where the occipital region is flat can be prevented.

Further, when the pressure is applied to the sides of the head and the occipital region except for the peak portion, the cranium of the infant can be naturally deformed toward the peak portion of the occipital region to which no pressure is applied. Therefore, the correction effect of the plagiocephaly can be more improved and the head of the infant can be formed into the scaphocephalic head shape.

In addition, as the empty space is formed between the peak portion of the occipital region and the soft inner cover 30, the air can be more effectively introduced into the cranial orthosis. That is, the ventilation can be improved and thus the sweat can be easily removed. Therefore, the wearing comfort can be improved.

In addition, as the protective outer cover 10 is formed in the semi-helmet shape and the head fixing member 40 and the jaw hooking member 50 for fixing the cranial orthosis on the head are further provided, the head of the infant can be corrected with a minimum size. Even when the size is reduced, the wearing state can be securely maintained. In addition, the user cannot feel the heavy pressure and the ventilation is improved, thereby improving the wearing comfort.

The above-described operational effects can be attained even when the infant lies obliquely as shown in FIG. 3 since the protective outer cover 10 is formed of the hard material and thus the shape thereof is maintained.

The following will describe a cranial orthosis according to a second embodiment. Since the second embodiment is similar to the first embodiment except for the structure of the soft inner cover. Therefore, the difference will be mainly described and like reference numbers will be used to refer to like parts.

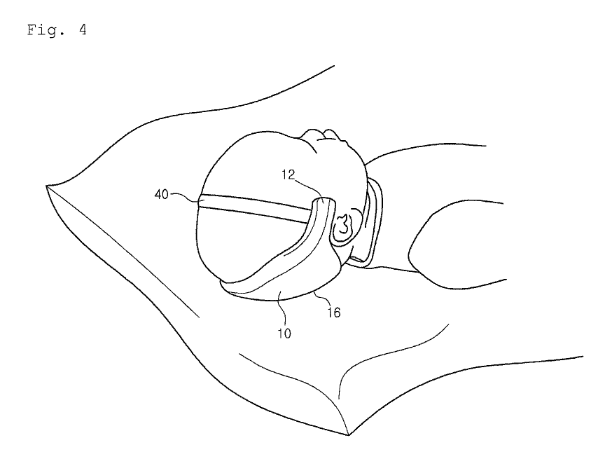

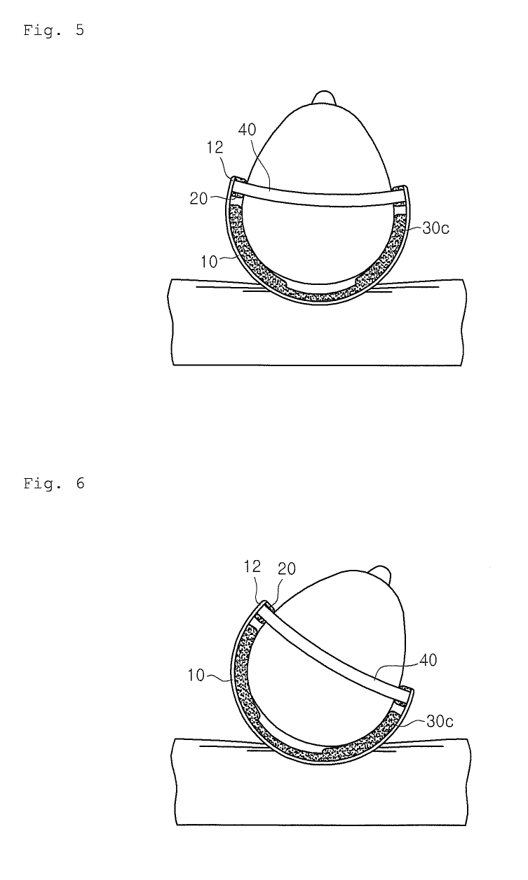

FIG. 5 is a view illustrating a state where an infant wearing a cranial orthosis according to a second embodiment lies on his/her back on a floor (mattress), and FIG. 6 is a view illustrating a state where the infant wearing the cranial orthosis of FIG. 5 lies obliquely.

Referring to FIGS. 5 and 6, both sides of a soft inner cover 30c of a cranial orthosis of the second embodiment are thicker than other portions.

In more detail, the soft inner cover 30c is monolithic and attached to an inner surface of a protective outer cover 10. The both sides of the soft inner cover 30c, which contact the sides of the head of the infant and sides of the occipital region of the infant are formed to be thicker than other portions. Accordingly, the pressure applied to the sides of the head and the sides of the occipital region may be further increased. In addition, an empty space between a peak portion of the occipital region of the infant and the soft inner cover 30c is further increased and thus the effectiveness of the cranial orthosis can be maximized.

The following will describe a cranial orthosis according to a third embodiment. Since the third embodiment is similar to the first embodiment except for the structure of the soft inner cover. Therefore, the difference will be mainly described and like reference numbers will be used to refer to like parts.

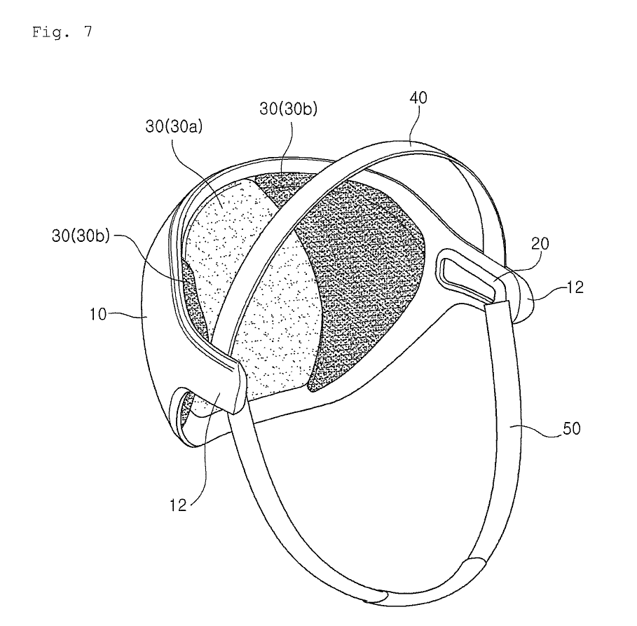

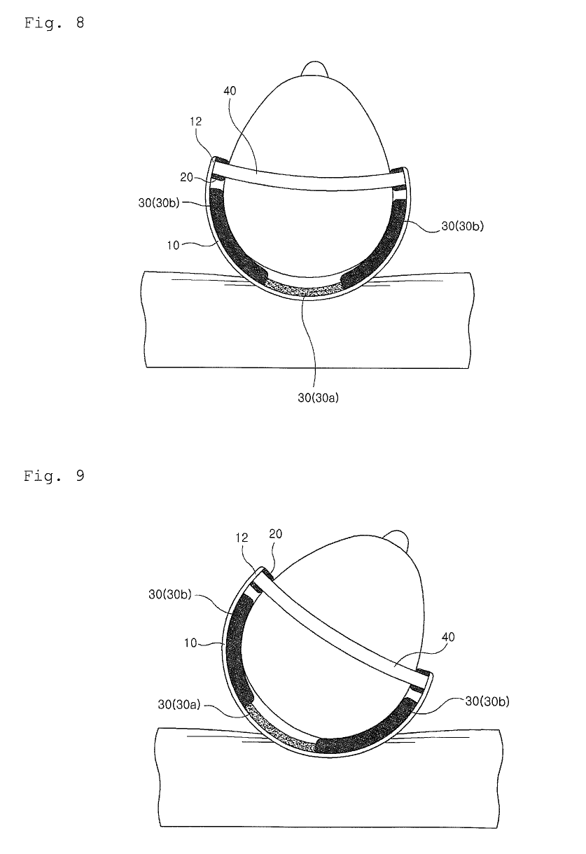

FIG. 7 is a perspective view of a cranial orthosis according to a third embodiment, FIG. 8 is a view illustrating a state where an infant wearing the cranial orthosis of FIG. 7 lies on his/her back on a floor (mattress), and FIG. 9 is a view illustrating a state where the infant wearing the cranial orthosis of FIG. 8 lies obliquely.

Referring to FIGS. 7 to 9, a soft inner cover 30 of a cranial orthosis of a third embodiment includes a plurality of sections such that a peak portion of the occipital region is more reliably spaced apart from the soft inner cover 30.

In more detail, the soft inner cover 30 includes side parts 30b that mainly contact and a middle part 30a provided between the side parts 30b and thinner than the side parts 30b.

In this case, an empty space is formed between a peak portion of the occipital region and the middle part 30a when the infant lies on his/her back and obliquely. Since the empty portion is structurally formed between the peak portion of the occipital region and the middle portion 30a, the peak portion is not directly pressed and thus the flattening of the occipital region can be prevented.

Further, as the sides of the head and the sides of the occipital region uniformly contact the side parts 30b that are thicker (higher) than the middle part 30a, the head shape of the infant is naturally induced into the scaphocephalic shape and the asymmetrical deformation of the cranium can be prevented.

Further, since the side parts 30b directly contact the head of the infant, the side parts 30b can absorb a greater amount of the sweat than the middle part 30a. Therefore, only the side parts 30b can be separated and washed and thus the cranial orthosis can be more conveniently used.

At this point, as a modified example of the third embodiment, the middle part 30a may be formed to contact the peak portion of the occipital region. In this case, the middle portion 30a simply contacts the peak portion but not applies any pressure to the peak portion. That is, the pressure applied to the peak portion by the middle part is substantially almost 0. That is, the substantially pressure is applied to the head of the infant by the side parts 30b and the middle portion 30a simply contacts the head to improve the wearing comfort.

The following will describe a cranial orthosis according to a fourth embodiment. Since the fourth embodiment is similar to the first embodiment except for the structures of the protective outer cover and the soft inner cover. Therefore, the difference will be mainly described and like reference numbers will be used to refer to like parts.

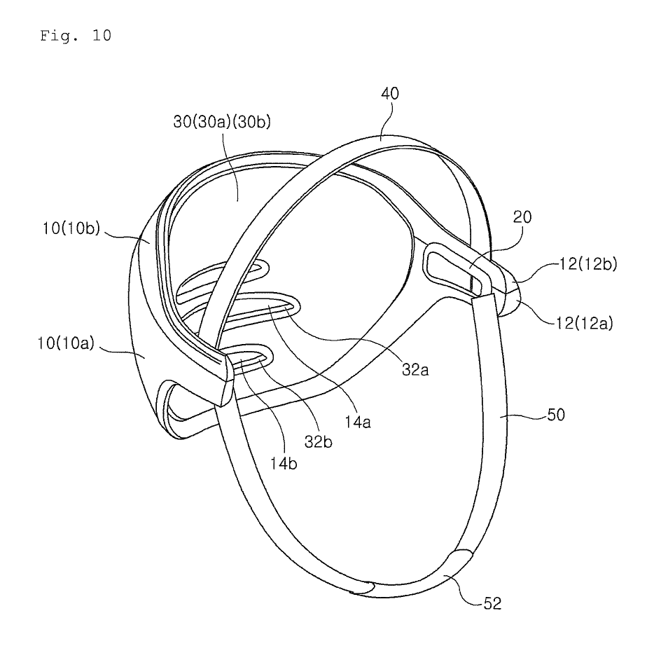

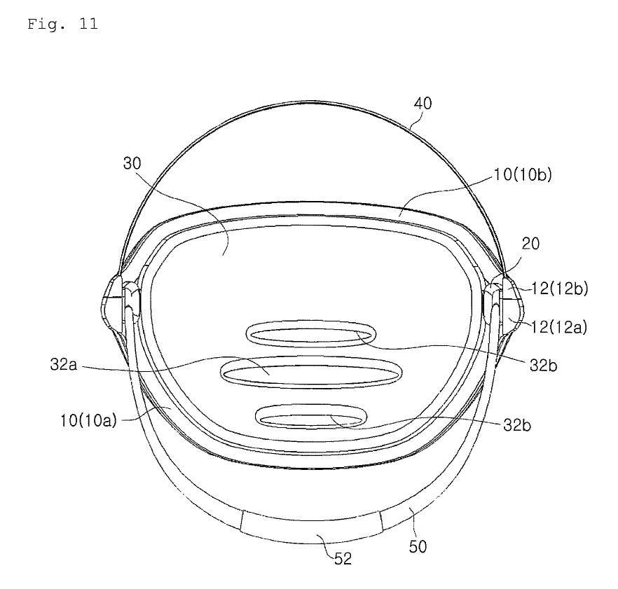

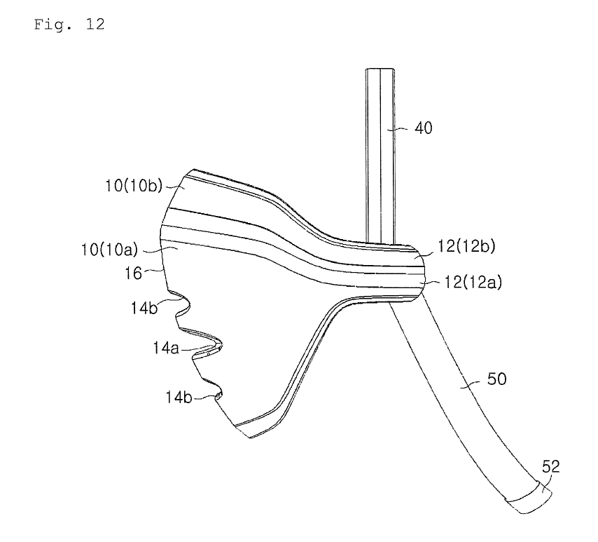

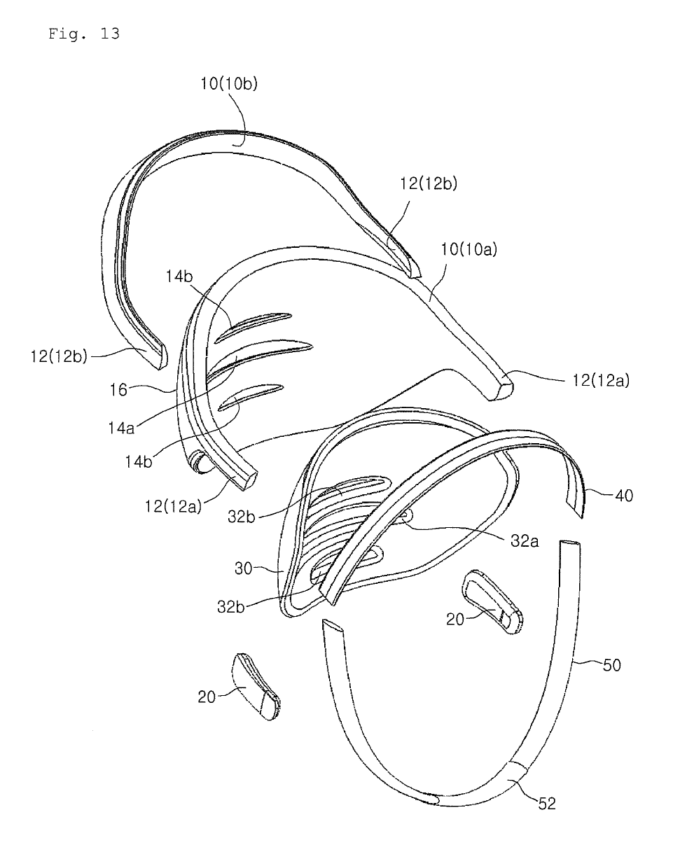

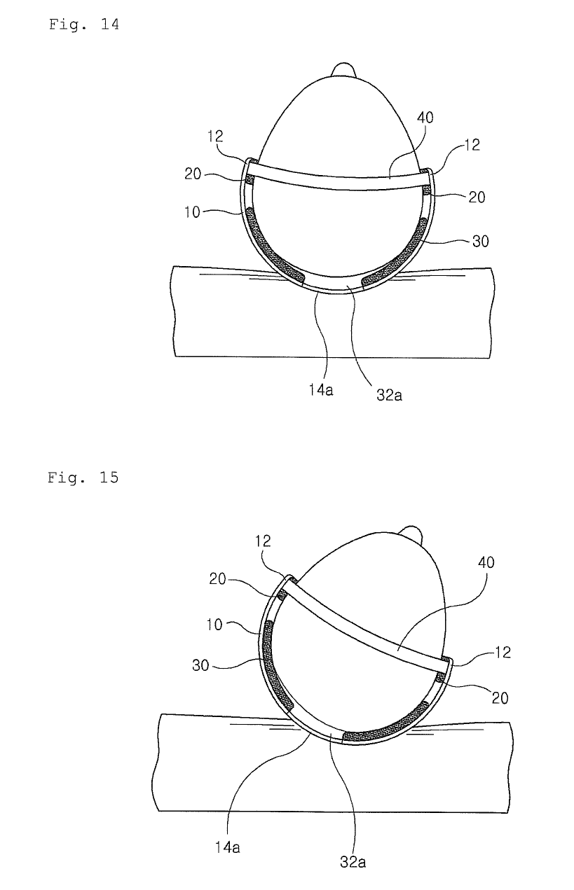

FIG. 10 is a perspective view of a cranial orthosis according to a fourth embodiment, FIG. 11 is a front view of the cranial orthosis of FIG. 10, FIG. 12 is a side view of the cranial orthosis of FIG. 10, FIG. 13 is a view illustrating a separating state of the cranial orthosis of FIG. 10, FIG. 14 is a view illustrating a state where an infant wearing the cranial orthosis of FIG. 10 lies on his/her back on a floor (mattress), and FIG. 15 is a view illustrating a state where the infant wearing the cranial orthosis of FIG. 14 lies obliquely.

Referring to FIGS. 10 to 15, according to a cranial orthosis of a fourth embodiment, a protective outer cover 10 is divided into upper and lower outer covers 10b and 10a considering a formability and design of a product. The upper and lower outer covers 10b and 10a can be separable coupled to each other.

In more detail, the protective outer cover 10 is designed such that the lower outer cover 10a is coupled to a lower portion of the upper outer cover 10b in a state where a head fixing member 40 is coupled to the upper outer cover 10b. At this point, each of the arm portions 12 is also divided into an upper section 12b and a lower section 12a that are formed protruding from the upper and lower outer covers 10b and 10a. A soft inner cover 30 is monolithic and attached to the inner surface of the protective outer cover 10.

According to the cranial orthosis of this fourth embodiment, a plurality of ventilation holes 14a, 14b, 32a, and 32b are formed through central portions of the protective outer cover 10 and soft inner cover 30. Therefore, when the infant wears the cranial orthosis, the air can be directly introduced and thus the ventilation can be improved.

In more detail, the ventilation holes 14a, 14b, 32a, and 32b are formed to be spaced apart from a peak portion of the occipital region. That is, since the air can be introduced into an empty space formed between the soft inner cover 30 and the peak portion and thus the ventilation effect can be maximized. Accordingly, the sweat and heat generated from the head can be effectively removed through the ventilation holes 14a, 14b, 32a, and 32b, thereby improving the wearing comfort.

In addition, as the ventilation holes 14a, 14b, 32a, and 32b are provided, the rear end portion of the protective outer cover 30 can be naturally flat. Therefore, when the infant lies on his/her back, the lying posture of the infant can be maintained.

At this point, the ventilation holes 14a, 14b, 32a, and 32b are formed in a rectangular or streamline shape so that the air can smoothly flow pass therethrough. The middle ventilation holes 14a and 32a may be wider than the upper and lower ventilation holes 14b and 32b. Needless to say, the number and shape of the ventilation holes 14a, 14b, 32a, and 32b are not limited to the above configuration.

According to the cranial orthosis of this fourth embodiment, an empty space is formed between the peak portion of the occipital region and the soft inner cover when the infant lies on his/her back and obliquely. Therefore, the fourth embodiment can attain the same effect as the first embodiment.

The following will describe a cranial orthosis according to a fourth embodiment. Since the fifth embodiment is similar to the first embodiment except for the structure of the soft inner cover. Therefore, the difference will be mainly described and like reference numbers will be used to refer to like parts.

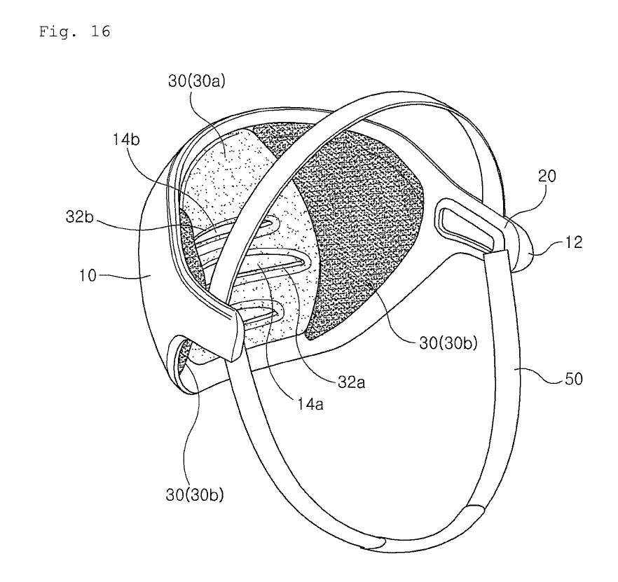

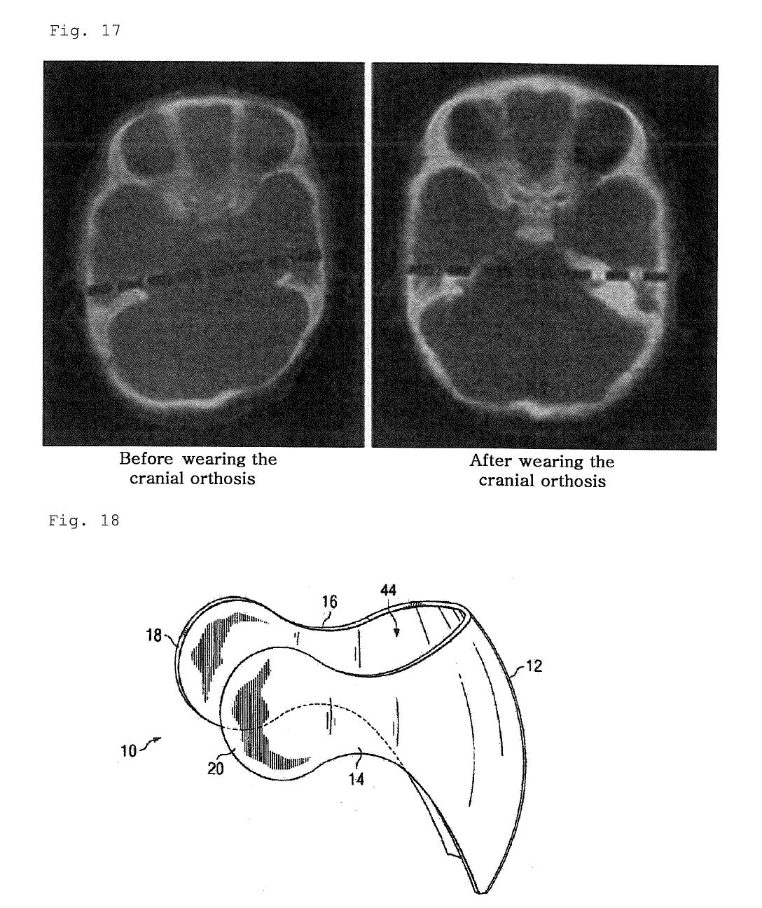

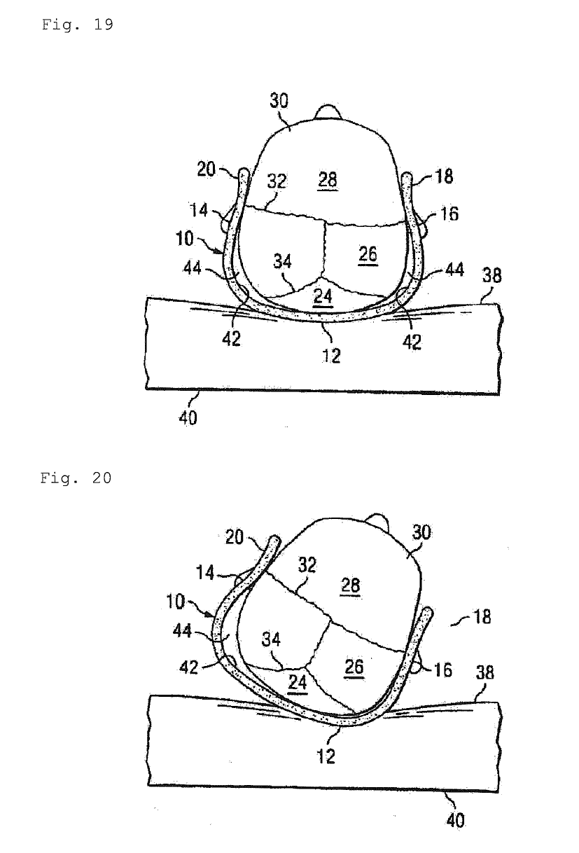

FIG. 16 is a perspective view of a cranial orthosis according to a fifth embodiment.

Referring to FIG. 16, a soft inner cover 30 of a cranial orthosis of a fifth embodiment includes a plurality of sections such that a peak portion of the occipital region is more reliably spaced apart from the soft inner cover 30.

In more detail, the soft inner cover 30 includes side parts 30b that mainly contact and a middle part 30a provided between the side parts 30b and thinner than the side parts 30b.

Therefore, the fifth embodiment can attain the same effects as the third and fourth embodiments.

According to the embodiments, since the protective outer cover formed of the hard material and the inner cover formed of the soft material are provided together to smoothly apply pressure to the sides of the head and sides of the occipital region except for the peak portion, the brachycephaly and plagiocephaly can be prevented and it is possible to correct the brachycephalic head into the scaphocephalic head.

Particularly, for the brachycephalic head that is long in the leftward-rightward direction and short in the frontward-backward direction, the forehead and rear head is wide and flat in the leftward-rightward direction and thus, when the cranial orthosis is used, the sides of the head and the sides of the occipital region except for the peak portion can uniformly contact the cranial orthosis, thereby improving the correction effect.

Further, since the inner cover is formed of the soft material and contacts the head of the infant, impact that may be applied to the head of the infant can be attenuated. Therefore, the cranial orthosis can be safely used in infants.

Specifically, since the pressure is not actively applied to the head of the infant by the shape of the outer cover but passively applied by the weight of the head of the infant, the secondary disease can be prevented.

Further, since the outer and inner covers are designed in the semi-helmet shape to enclose only the sides of the head and sides of the occipital region, the burden caused by the weight of the helmet can be reduced and the heavy pressure feeling by the wearer can be reduced. In addition, ventilation is improved and thus the wearing comfort can be improved.

In addition, since the wearer can sleep soundly, there is no need to put the infant sleep on his/her stomach to correct the head shape. Therefore, the sudden death of the infant during the sleeping can be prevented.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination within the scope of the disclosure, the drawings and the appended claims. In addition to variations and modifications in the component parts and/or arrangements, alternative uses will also be apparent to those skilled in the art.

What is claimed is:

1. A cranial orthosis comprising:
a protective outer cover that is formed of a hard material and having an inner surface formed in a scaphocephalic shape to cover at least an occipital region and sides of a head; and
a soft inner cover that is formed of a soft material, shaped to correspond to the protective outer cover, and mounted on an inner surface of the protective outer cover,
wherein the soft inner cover is formed to be spaced apart from a peak portion of the occipital region.

2. The cranial orthosis according to claim 1, wherein the inner surface of the protective outer cover is formed in an oval-shaped normal scaphocephalic head shape.

3. The cranial orthosis according to claim 1, wherein the soft inner cover is formed to contact the sides of the head and side of the occipital region except for the peak portion.

4. The cranial orthosis according to claim 3, wherein the contact portion of the soft inner cover is thicker than other portions.

5. The cranial orthosis according to claim 1, wherein a ventilation hole is formed through the protective outer cover and the soft inner cover at a location spaced apart from the occipital region.

6. The cranial orthosis according to claim 1, wherein the soft inner cover comprises:
a middle part that does not contact the head; and
side parts provided at both sides of the middle part and contacting at least the sides of the head and sides of the occipital region.

7. A cranial orthosis comprising:
a protective outer cover that is formed of a hard material and having an inner surface formed in a scaphocephalic shape to cover at least an occipital region and sides of a head; and
a soft inner cover that is formed of a soft material, shaped to correspond to the protective outer cover, and mounted on an inner surface of the protective outer cover,
wherein the soft inner cover is formed to apply relatively higher pressure to sides of the head and sides of the occipital region,
and the pressure is generated by weight of a wearer's head.

8. The cranial orthosis according to any one of claims 1 and 7, further comprising:
arm portions extending frontward from both ends of the protective outer cover;
a head fixing member formed above the protective outer cover and closely contacting a top of the head to prevent the cranial orthosis from moving downward; and
a jaw hooking member formed under the protective outer cover and hooked around a jaw to prevent the cranial orthosis from moving upward.

9. The cranial orthosis according to claim 8, wherein the head fixing member and the jaw hooking member are provided in the form of an elastic band.

10. The cranial orthosis according to any one of claims 1 and 7, wherein a rear end portion of the protective outer cover is flat.

11. The cranial orthosis according to any one of claims 1 and 7, wherein the protective outer cover is formed by a plurality of sections that are coupled to each other.
